(19) United States
(12) Patent Application Publication
(10) Pub. No.: US 2004/0170587 A1
(43) Pub. Date: Sep. 2, 2004

(54) Method of compatibilizing cationic materials with anionic polymers

(76) Inventor: Brian Jay Vondruska, Maple Heights, OH (US)

Correspondence Address: NOVEON IP HOLDINGS CORP., 9911 BRECKSVILLE ROAD, CLEVELAND, OH, 44141-3247, US

(21) Appl. No.: 10/792,993

(22) Filed: Mar. 4, 2004

Related U.S. Application Data
(63) Continuation-in-part of application No. 10/653,609, filed on Sep. 2, 2003.
(60) Provisional application No. 60/408,793, filed on Sep. 6, 2002.

(51) Int. Cl. A61K 7/06; A61K 7/11

(52) U.S. Cl. 424/70.12

(57) ABSTRACT

A method of compatibilizing an anionic polymer with cationic materials, which comprises complexing a cationic material with a compatible anionic complexing agent before combining the complexed cationic material with an anionic polymer. A composition comprising an anionic polymer and a complexed cationic material and a composition, such as a coating, containing an anionic polymer and a cationic material complexed with an anionic complexing agent.

### CROSS REFERENCE TO RELATED APPLICATIONS

This application is a Continuation-in-Part of U.S. Ser. No. 10/653,609 filed Sep. 2, 2003, which claims the benefit of U.S. Provisional Application Serial No. 60/408,793 filed Sep. 6, 2002. The disclosure of said Ser. No. 10/653,609 is hereby incorporated by reference.

### BACKGROUND OF THE INVENTION

Anionic polymers are employed in many end-use applications. Rheology modifiers (thickeners), for example, are generally employed in most personal care products and other products of that nature. Some of the most useful anionic polymers are based on ethylenically unsaturated carboxylic acid monomers which includes crosslinked polyacrylic acid or copolymers of ethylenically unsaturated carboxylic acid monomers and copolymerizable vinyl monomers. Such polymers yield anionic polymers that are extremely useful in various personal care products in the cosmetic and toiletry industries as well as in a variety of other applications, such as coatings (paper, textiles, nonwovens, fibers) and others.

In addition to anionic polymers, such products generally require a variety of other materials, especially cationic materials. Often cationic materials, such as cationic surfactants, are not compatible with anionic polymers. G. Polotti and F. code in “Thickener for Cationic Surfactant Solutions” in the Proceedings of the 28th CED Annual Meeting, Barcelona, Spain, 1998, stated: “The thickening of cationic surfactant solutions is often a challenging problem in the detergent industry especially for the formulation of fabric softeners, toilet bowl cleaners, lime scale removers, etc. Part of the problem comes because the most common thickeners, such as those based on crosslinked polyacrylic acid, are anionic species. Although stable and viscous suspensions are achievable, the combination of polyacrylic acid and cationic surfactants forms aggregates that cannot be shared in further dilution. The effect of the cationic species is consequently lost in the strong bond with the anionic materials.”

In the Handbook of Cosmetic Science and Technology, First Edition 1993 Elsevier Science Publishers Ltd., on page 17, it is stated:

“Carbomers are incompatible with cationic surfactants and show a significant reduction in viscosity building potential in the presence of electrolytes. For this reason, their use in the stabilization of detergent-based products is very limited.”

Consequently, there is a great need for compatibilizing anionic polymers with cationic surfactants or other cationic materials, including cationic polymers.

There are several U.S. patents or published patent applications that disclose the use of rheology modifiers and silicones in various cosmetic or personal care compositions.

U.S. Pat. No, 4,210,161 discloses a cream rinse composition comprising an anionic polymer and a cationic surfactant capable of forming a water insoluble reaction product. Thus, this patent clearly states that the anionic polymer and a cationic surfactant are incompatible and do form a precipitate but in this formulation, such a precipitate is desirable.

U.S. Pat. No. 4,710,374 discloses cosmetic compositions containing a cationic polymer and an anionic polymer latex. The patent disclosure clearly stresses that the cationic polymer is of a relatively high molecular weight of between 500 to 3,000,000 but most, if not all, appear to be at least 10,000 molecular weight and more often, about 500,000 molecular weight. Thus, the cationic ingredient is a large molecule with a low charge density. For this reason, the cationic polymer and the anionic polymeric latex are not truly incompatible.

U.S. Pat. No. 6,071,499 discloses cosmetic compositions with an anionic acrylic polymer and an oxyalkylenated silicone which is nonionic. Since the silicone is not anionic, it cannot complex with a cationic ingredient although it is said to improve the performance of such anionic polymer.

Published U.S. application Ser. No. 2003/0108503 A1 discloses a composition comprising a copolymer of methacrylic acid and an alkyl acrylate, a cationic or amphoteric polymer and a functionalized silicone. Apparently, the disclosed anionic polymers are compatible with the disclosed cationic polymeric surfactants. The three components are combined together without first forming a complex of a cationic polymer with the functionalized silicone. Consequently, no compatibilization or complex formation is involved in the invention disclosed in the published application.

### SUMMARY OF THE INVENTION

The invention is directed to a method of compatibilizing anionic polymers with cationic materials, such as cationic surfactants, cationic polymers or cationic salts, which method comprises complexing the cationic material with an anionic complexing agent before combining the complexed cationic material with an anionic polymer. The invention is further directed to a composition comprising an anionic polymer and a complexed cationic material and to compositions containing an anionic polymer and a cationic material complexed with an anionic complexing agent.

Since many, though not all, cationic materials are incompatible with anionic polymers, when they are combined usually a precipitate forms, or turbidity develops and some of the properties, such as thickening, of the anionic polymers are generally substantially altered. By first complexing the cationic material(s) with an anionic complexing agent before combining with an anionic polymer, the incompatible anionic polymer thickeners and the cationic materials become compatibilized. When such compatibilized cationic material(s) is combined with an anionic polymer, the viscosity/turbidity profile of the resulting compositions is substantially improved. Thus, the complexing of the cationic materials prior to combining them with anionic polymers either reduced or eliminated excessive turbidity and the tendency to form precipitates.

### DETAILED DISCLOSURE

The truly unexpected feature of the present invention is the fact that the cationic materials, which generally are not compatible with anionic polymers, can be made compatible by first complexing them with an anionic compatibilizing agent without negatively affecting the performance and function of the cationic materials. The cationic materials that may be compatibilized with anionic polymers are quaternary ammonium salts, polyquatemary ammonium salts, organic or inorganic salts, alkyl amines, amidoamines, ethoxylated amines, alkyl imidazolines and various cationic polymers. By “incompatible”, is meant that when such cationic materials are combined with anionic polymers, either a precipitate forms or turbidity develops.

When cationic materials are added to a formulation containing an anionic polymer, generally a precipitate and/or turbidity develops which is unacceptable for many products, such as personal care, coatings, household products or in other uses. This long existing difficulty, however, can be overcome and such materials can be compatibilized by the instant invention, wherein cationic materials are first complexed with a compatibilizing agent which is an anionic bulky molecule containing an anionic group such as a sulfate, sulfonate, phosphate, phosphonate or carboxylate groups. By “compatibilized” is meant elimination or at least a substantial reduction of the formation of a precipitate or turbidity that would otherwise result. By “substantial reduction” is meant a reduction to such a degree that such materials (the cationic materials and the anionic polymers) can be successfully employed in a particular end-use product. Generally, such reduction would constitute at least a 50% reduction of turbidity formation and preferably at least 80% reduction, such that the turbidity of compositions or formulations containing both cationic material(s) and anionic polymer(s) is not greater than 50, often 20 NTU and preferably 15 NTU or less. In clear gels or coatings, it is preferable that the turbidity be 15 NTU or less and preferably 10 NTU or less. The level of turbidity that is considered acceptable always depends on the end-use application. The use of complexed anionic materials of this invention also aid in efficient use of anionic polymers by often enabling the use of a lesser amount of an anionic polymer yet obtaining desirable properties, thus making the resulting products more cost efficient. There should be practically a complete elimination of precipitate formation.

Generally, the cationic materials are not compatible with the anionic polymers. However, if the concentration of a cationic material is low enough, they may be compatible. Similarly, if the charge density is low enough (e.g. the charge moiety(s) is dispersed sparcely throughout the molecule) they may also be compatible. Consequently, this invention deals with compatibilizing cationic materials that are incompatible with the anionic polymers.

Cationic Materials

Cationic materials are commonly used as surfactants and as conditioning materials. Quaternary ammonium compounds (i.e. quats) are the most widely used of the many available classes of cationic materials. Their favorable safety profile, cost-effectiveness and long-term stability are additional factors. Polyquats are the polymeric counterparts of quats and are used in the same manner as quats, and for the same general purposes. They have additional utility as fixatives and rheology modifiers, due to their high molecular weight. Any cationic materials that are incompatible with any anionic polymers may be made compatible employing the present invention.

Illustrative, but non-limiting, examples of cationic materials are listed below.

A. Polyguaterniums

Hexadimethrine Chloride

Hydroxypropyl Guar Hydroxypropyltrimonium Chloride

Locust Bean Hydroxypropylthemonium Chloride

Polyacrylamidopropyltrimonium Chloride

Polymethacrylamidopropyltrimonium Methosulfate

Polyquaterium-1* to 20*, 22*, 24*, 27* to 37*, 39*, 42* to 50*

B. Monosubstituted Quaternaries

Basic Red 118*

Behenoyl PG-Trimonium Chloride

Behentrimonium Chloride

Behentrimonium Methosulfate

Benzyl Triethyl Ammonium Chloride

Bis-Hydroxyethyl Cocomonium Nitrate

Bis-Hydroxyethyl Dihydroxypropyl Stearammonium Chloride

Bis-Hydroxyethyl Rapeseedmonium Chloride

Bis-Hydroxyethyl Tallowmonium Chloride

B. Monosubstituted Quaternaries (Cont'd)

Camphor Benzalkonium Methosulfate

Carpronium Chloride

Ceteartrimonium Chloride

Cetrimonium Bromide, Chloride, Methosulfate, Saccharinate and Tosylate

Cetyl Ethyldimonium Ethosulfate

Coco-Ethyldimonium Ethosulfate

Cocotrimonium Chloride and Methosulfate

C4-18 Perfluoralkylethyl Thiohydroxypropyltrimonium Chloride

Dextran Hydroxypropyltrimonium Chloride

Dimethicone Hydroxypropyl Trimonium Chloride

Dodecylbenzyltrimonium Chloride

Dodecylhexadecyltrimonium Chloride

Dodecylxylylditrimonium Chloride

Galactoarabinan Hydroxypropyltrimonium Chloride

Ginsing Hydroxpropyltrimonium Chloride

Guar Hydroxpropyltrimonium Chloride

Hydrogenated Tallowtrimonium Chloride

Hydroxypropyl Bistrimonium Diiodide

Hydroxypropyltrimonium Chloride

Hydroxypropyltrimonium Honey

Hydroxypropyltrimonium Hydrolyzed Whey

Isostearoyl PG-Trimonium Chloride

Isostearyl Ethyldimonium Chloride

Lactamidopropyl Trimonium Chloride

Lauroyl PG-Trimonium Chloride

Laurtrimonium Bromide, Chloride and Trichlorophenoxide

Octyldodecyltrimonium Chloride

Oleamine Bishydroxypropyltrimonium Chloride

Oleoyl PG-Trimonium Chloride

Palmitamidopropyltrimonium Chloride

Palmitoyl PG-Trimonium Chloride

PEG-1 and PEG-10 Coco-Benzonium Chloride

PEG-2 and PEG-15 Cocomonium Chloride

PEG-5 Cocomonium Methosulfate

PEG-2 and PEG-15 Oleammonium Chloride

PEG-2 and PEG-15 Stearmonium Chloride

PEG-5 Stearyl Ammonium Chloride and Lactate

PEG-20 Tallow Ammonium Ethosulfate

PEG-5 Tallow Benzonium Chloride

PPG-9, PPG-25 and PPG-40 Diethylmonium Chloride

Quaternium-16*, 22*, 26*, 30*, 33*, 52*, 60*, 61*, 75* and 88*

Soytrimonium Chloride

Stearoyl PG-Trimonium Chloride

Steartrimonium Bromide

B. Monosubstituted Quaternaries (Cont'd)

Steartrimonium Methosulfate

Steartrimonium Sacchannate

Tallow Trihydroxyethylammonium Acetate

Tallowtrimonium Chloride

C. Disubstituted Quaternaries

Behenalkonium Chloride

Benzalkonium Bromide and Chloride

Benthethonium Bromide or Chloride

Benzalkonium Cetyl Phosphate

Benzoxonium Chloride

C12-18 Dialkyldemonium Chloride

Cetalkonium Chloride

Cetearalkonium Bromide

Cetethyldimonium Bromide

Cetethyl Morpholinium Ethosulfate

Cetyl Pyrrolidonylmethyl Dimonium Chloride

Cocoalkonium Chloride

Denatonium Benzoate and Saccharide

Dibehenyl/Diarachidyl Dimonium Chloride

Dibehenyldimonium Chloride and Methosulfate

Di-C12-15, C12-18 and C14-18 Alkyl Dimonium Chloride

Dicetyldimonium Chloride

Dicocodimonium Chloride

Dicocoylethyl Hydroxyethylmonium Methosulfate

Didecyldimonium Chloride

Dihydrogenated Palmoylethyl Hydroxyethylmonium Methosulfate

Dihydrogenated Palmoyl Hydroxyethylmonium Methosulfate

Dihydrogenated Tallow Benzylmonium Chloride and Hectorite

Dihydrogenated Tallowethyl Hydroxyethylmonium Methosulfate

Dihydrogenated Tallow Hydroxyethylominium Methosulfate

Dihydrogenated Tallow Hydroxyethylmonium Methosulfate

Dihydrogenated Tallowayethyl Hydroxyethylmonium Methosulfate

Dihydroxpropyl PEG-5 Linoleammonium Chloride

Diisostearamidopropyl Epoxypropylmonium Chloride

Dilaureth-4 Dimonium Chloride

Dilauryl Acetyl Dimonium Chloride

Dilauryldimonium Chloride

Dimethyl PABA Ethyl Cetearyldimonium Tosylate

Dimethyl PABA Midopropyl Laurdimonium Tosylate

Dioleoylamidoethyl Hydroxyethylmonium Methosulfate

Dioleoyl Edetolmonium Methosulfate

Dioleoyl EDTHP Monium Methosulfate

C. Disubstituted Quaternaries (Cont'd)

Dipalmitoylethyl Dimonium Chloride

Dipalmitoylethyl Hydroxyethylmonium Methosulfate

Dipalmoylethyl Hydroxyethylmonium Methosulfate

Dipalmoylisopropyl Dimonium Methosulfate

Disoydimonium Chloride

Disoyoylethyl Hydroxyethylmonium Methosulfate

Disteardimonium Hectorite

Disteareth-6 Dimonium Chloride

Distearoylethyl Dimonium Chloride

Distearoylethyl Hydroxyethylmonium Methosulfate

Distearoylpropyl Trimonium Chloride

Distearyldimonium Chloride

Distearyl Epoxypropylmonium Chloride

Ditallowamidoethyl Hydroxypropylmonium Methosulfate

Ditallow Dimonium Cellulose Sulfate

Ditallowdimonium Chloride

Ditallowoylethyl Hydroxyethylmonium Methosulfate

Ditridecyldimonium Chloride

Domiphen Bromide

Erucalkonium Chloride

Hydrogenated Tallowalkonium Chloride

Hydroxycetyl Hydroxyethyl Dimonium Chloride

Hydroxyethyl Cetyldimonium Chloride and Phosphate

Hydroxyethyl Laurdimonium Chloride

Hydroxyethyl Tallowdimonium Chloride

Hydroxypropyl Biscetearyldimonium Chloride

Hydroxypropyl Bisoleyldimonium Chloride

Hydroxypropyl Bisstearyldimonium Chloride

Isostearyl Laurdimonium Cloride

Lauralkonium Bromide and Chloride

Lauryl Methyl Gluceth-10 Hydroxypropyldimonium Chloride

Methylbenzethonium Chloride

Myristaklonium Chloride, Bromide and Saccharinate

Olealkonium Chloride

Oleoyl Epoxypropyldimonium Chloride

Panthenyl Hydroxypropyl Steardimonium Chloride

PEG-9 and 25 Diethylmonium Chloride

PEG-2 Dimeadowfoamamidoethylmonium Methosulfate

PEG-3 Dioleoylamidoethylmonium Methosulfate

PEG-5 Ditridecylmonium Chloride

PEG-8 Palmitoyl Methyl Diethonium Methosulfate

PEG-10 Stearyl Benzonium Chloride

PEG-3 Tallow Propylenedimonium Dimethosulfate

Quaternium-8*, 14*, 18*, 24*, 43*, 53*, 63*, 70*, 71* and 84*

C. Disubstituted Quaternaries (Cont'd)

Quaternium-18 Bentonite*

Quaternium-18 Benzalkonium Bentonite

Quaternium-18 Hectorite* and Methosulfate*

Sodium Coco PG-Dimonium Chloride Phosphate

Soy Dihydroxypropyldimonium Glucoside

Soydimonium Hydroxypropyl Hydrolyzed Wheat Protein

Soyethyldimonium Ethosulfate

Stearalkonium Bentonite, Chloride and Hectorite

Stearyl Ethylhexyldimonium Chloride and Methosulfate

Stearyl PG-Dimonium Chloride Phosphate

Tallowalkonium Chloride

Tallowdimonium Propyltrimonium Dichloride

Thiamine Diphosphate

D. Tetrasubstituted Quaternaries

Quaternium-15 *

Tetrabutyl Ammonium Bromide

Tetramethylammonium Chloride

E. Heterocyclic Quaternaries

Cetylpyridinium Chloride

Cocoyl Benzyl Hydroxyethyl Imidazolinium Chloride

Cocoyl Hydroxyethylimidazolinium PG-Chloride Phosphate

Dequalnium Acetate and Chloride

Dimethylaminostyrol Heptyl Methyl Thiazolium Iodide

Hydroxyanthraquinoneaminopropyl Methyl Morpholinium Methosulfate

Isostearyl Benzylimidonium Chloride

Isostearyl Ethylimidazolinium Ethosulfate

Lapyrium Chloride

Lauryl Isoquinolinium Bromide and Saccharinate

Laurylpyridinium Chloride

Platonin*

Quaternium-27*, 45*, 51*, 56*, 72*, 73*, 83* and 87*

Soyethyl Morpholinium Ethosulfate

Stearyl Hydroxyethylimidonium Chloride

Tall Oil Benzyl Hydroxyethyl Imidazolinium Chloride

F. Substituted Amido Quaternaries

Acetamidoethoxybutyl Trimonium Chloride

Acetamidopropyl Trimonium Chloride

Acrylamedopropyltrimonium Chloride/Acrylamide Copolymer

Acrylamidopropyltrimonium Chloride/Acrylates Copolymer

Almondamidopropalkonium Chloride

Apricotamidopropyl Ethyldimonium Ethosulfate

F. Substituted Amido Quaternaries (Con'd)

Avocadamidopropalkonium Chloride

Babassuamidopropalkonium Chloride

Behenamidopropyl Ethyldemonium Ethosulfate

Behenamidopropyl PG-Dimonium Chloride

Canolamidopropyl Ethyldimonium Ethosulfate

Carboxymethyl Isostearamidopropyl Morpholine

Cinnamidopropyltrimonium Chloride

C14-20 and C18-22 Isoalkylamidopropylethyldimonium Ethosulfate

Cocamidopropyl Betaine MEA Chloride

Cocamidopropyldimonium Hydroxypropyl Hydrolyzed Collagen

Cocarnidopropyl Ethyldimonium Ethosulfate

Cocamidopropyl PG-Dimonium Chloride and Chloride Phosphate

Cocamidopropyltrimonium Chloride

Dihydrogenated tallowamidoethyl Hydroxyethylmonium Chloride and Methosulfate

Hydroxypropyl Bisisostearamidopropyldimonium Chloride

Hydroxystearamidopropyl Trimonium Chloride

Hydroxystearamedopropyl Trimonium Methosulfate

Isononamidopropyl Ethyldimonium Ethosulfate

Isostearamidopropyl Epoxypropyl Dimonium Chloride

Isostearamidopropyl Epoxypropylmorpholinium Chloride

Isostearamidopropyl Ethyldimonium Ethosulfate

Isostearamidopropyl Ethylmorpholinium Ethosulfate

Isostearamidopropyl Laurylacetodimonium Chloride

Isostearamidopropyl PG-Dimonium Chloride

Isostearamenopropalkonium Chloride

Isostearyl Behenamidopropyl Betainate

Isostearyl Dilinoleamidopropyl Betainate

Isostearyl Racinoleamidopropyl Betainate

Methylene bis (tallowacetamiddimonium Chloride)

Lauramidopropyl Acetamidodimonium Chloride

Lauramidopropyl PG-Dimonium Chloride

Linoleamidopropyl Ethyldimonium Ethosulfate

Linoleamidopropyl PG-Dimonium Chloride Phosphate and Phosphate Dimethicone

Minkamidopropalkonium Chloride

Minkamidopropyl Ethyldimonium Ethosulfate

Oleamidopropyldimonium Hydroxypropyl Hydrolyzed Collagen

Oleamidopropyl Ethyldimonium Ethosulfate

Oleamidopropyl PG-Dimonium Chloride

Rapeseedamidopropyl Benzyldimonium Chloride

Rapeseedamidopropyl Epoxypropyl Dimonium Chloride

Rapeseedamidopropyl Ethyldimonium Ethosulfate

Ricebranamidopropyl Hydroxyethyl Dimonium Chloride

F. Substituted Amido Quaternaries (Con'd)

Ricinoleamidopropyl Ethyldimonium Ethosulfate

Ricinoleamidopropyltrimonium Chloride and Methosulfate

Saffloweramidopropyl Ethyldimonium Ethosulfate

Sodium Borageamidopropyl PG-Dimonium Chloride Phosphate

Sodium Emuamidopropyl PG-Dimonium Chloride Phosphate

Sodium Milkamidopropyl PG-Dimonium Chloride Phosphate

Sodium Oleamidopropyl PG-Dimonium Chloride Phosphate

Sodium Sunfloweramidopropyl PG-Dimonium Chloride Phosphate

Soyamidoethyldimonium/Trimonium Hydroxypropyl Hydrolyzed Wheat Protein

Soyamidopropalkonium Chloride

Soyamidopropyl Ethyldimonium Ethosulfate

Stearamidopropalkonium Chloride

Stearamidopropyl Cetearyl Dimonium Tosylate

Stearamidopropyl Ethyldimonium Ethosulfate

Stearamidopropyl PG-Dimonium Chloride Phosphate

Stearamidopropyl Pyrrolidonylmethyl Dimonium Chloride

Stearamidopropyl Trimonium Methosulfate

Undecylenamidopropyltrimonium Methosulfate

Wheat Germamidopropalkonium Chloride

Wheat Germamidopropalkonium Hydroxypropyl Hydrolyzed Wheat Protein

Wheat Germamidopropyl Epoxypropyldimonium Chloride

Wheat Germamidopropyl Ethyldimonium Ethosulfate

G. Quaternized Keratin

AMP-Isostearolyl Gelatin/Keratin Amino Acids/Lysine

Cocodimonium Hydroxypropyl Hydrolyzed Hair Keratin and Keratin

Hydroxypropyltrimonium Gelatin and Hydrolyzed Keratin

Lauryldimonium Hydroxypropyl Hydrolyzed Keratin

Quaternium-79 Hydrolyzed Keratin*

Steardimonium Hydroxypropyl Hydrolyzed Keratin

H. Quaternized Collagen

Benzyltrimonium Hydrolyzed Collagen

Cocodimonium Hydroxypropyl Hydrolyzed Collagen

Hydroxypropyltrimonium Hydrolyzed Collagen

Lauryldimonium Hydroxypropyl Hydrolyzed Collagen

Propyltrimonium Hydrolyzed Collagen

Quaternium-76 and 79 Hydrolyzed Collagen*

Steardimonium Hydroxypropyl Hydrolyzed Collagen

Steartrimonium Hydroxyethyl Hydrolyzed Collagen

Triethonium Hydrolyzed Collagen Ethosulfate

I. Quaternized Amino Acids

Cocodimonium Hydroxypropyl Silk Amino Acids

Gelatin/Keratin Amino Acids/Lysine Hydroxypropyltrimonium Chloride

J. Quaternized Proteins

Cocodimonium Hydroxypropyl Hydrolyzed Casein, Silk, Rice Protein, Soy Protein & Wheat Protein

Gelatin/Lysine/Polyacrylamide Hydroxypropyltrimonium Chloride

Hydroxypropyltrimonium Hydrolyzed Casein and Conchiolin Protein

Hydroxypropyltrimonium Hydrolyzed Rice Bran Protein, Silk, Vegetable Protein,

Wheat Protein, Wheat Protein/Siloxysilcate

Laurdimonium Hydroxypropyl Hydrolyzed Soy Protein and Wheat Protein/Siloxysilicate

Lauryldimonium Hydroxypropyl Hydrolyzed Casein, Silk and Soy Protein

Propyltrimonium Hydrolyzed Soy Protein and Wheat Protein

Quaternium-79 Hydrolyzed Milk Protein*, Silk*, Soy Protein* and Wheat Protein*

Quaternium-86*

Steardimonium Hydroxypropyl Hydrolyzed Casein, Rice Protein, Silk, Soy Protein and Vegetable Protein

Steardimonium Hydroxypropyl Wheat Protein

K. Salts of Divalent or Polyvalent Cations

Aluminum Acetate and Acetate Solution

Aluminum Benzoate, Butoxide, Citrate, Diacetate, Dicetyl Phosphate, Lactate, Methionate, PCA, Sucrose Octasulfate and Triformate

Aluminum/Magnesium Hydroxide Stearate

Antimony Potassium Tartrate

Barium Gluconate

Bismuth Citrate and Subgallate

Brucine Sulfate

Calcium Acetate, Ascorbate, Benzoate, Citrate, Cyclamate, DNA,

Fructoheptonate, Glucoheptonate, Gluconate, Glycerophosphate, Lactate, Pantetheine Sulfonate,Pantothenate, Paraben, Propionate, Saccharine, Salicylate, Sorbate, Stearoyl Lactylate, Tartarate and Thioglycolate

Calcium Disodium EDTA

Cobalt Gluconate

CopperDNA, Gluconate, PCA, PCA Methylsilanol, Picolinate and Usnate

Cupric Acetate

Feric Ammonium Citrate

Ferric Citrate and Glycerophosphate

Ferrous Aspartate, Aglucoheptonate and Gluconate

Iron Picolinate

Isopropyl Titanium Triisostearate

K. Salts of Divalent or Polyvalent Cations (Cont'd)

Lead Acetate

Magnesium Acetate, Ascorbate, Ascorbate/PCA, Ascorbyl Phosphate, Benzoate, Citrate, DNA, Glucohiptonate, Gluconate, Glyerophosphate, PCA, Propionate, Salicylate and Thioglycolate

Magnesium Laureth-11 Carboxylate

Manganese Gluconate

Manganese Glycerophosphate

Manganese PCA

Molybdenum Aspartate

Nickel Gluconate

Phenyl Mercuric Acetate, Benzoate, Borate and Chloride

Strontium Acetate

Strontium Thioglycolate

Zinc Acetate, Citrate, Cysteinate, Dibutyldithiocarbamate, Glucoheptonate, Gluconate, Glycyrrhelinate, Lactate, Picolinate and Pyrithione

Zinc Formaldehyde Sulfoxylate

Zinc PCA

L. Pigments

Zinc Oxide, Iron Oxides, Titanium Dioxide

M. Organic Amines

Alanine Glutamate

Allantoin Acetyl Methionine, Ascorbate, Biotin, Calcium Pantothenate, Galacturonic Acid, Glycyrrhetinic Acid, PABA and Polygalacturonic Acid

Amodimethicone Hydroxystearate

Arginine Aspartate, DNA and PCA

Arginine Glutamate

Arginine Hexyldecyl Phosphate

Chitosan Adipate, Ascorbate, Glycolate and Salicylate

Chloramine T

Chlorhexidine Diacetate, Digluconate and Dihydrochloride

Chlorophyllin-Copper Complex

Ciclopirox Olamine

Cysteamine HCI

Cysteine DNA

DEA-Cetyl Phosphate

DEA-Hydrolyzed Lecithin

DEA-Methoxycinnamate

Dibehenamidopropyldimethylamine Dilinoleate

Dibromopropamidine Diisethionate

Diglycol Guanidine Succinate

Dihydroxyethyl Tallowamine Oleate

M. Organic Amines (Cont'd)

Dilithium Oxalate

Dimethicone Propylethylenediamine Behenate

Ethanolamine Dithiodiglycolate, Glycerophosphate and Thioglycolate

Ethyl Hydroxy Picolinium Lactate

Ethyl Lauroyl Arginate HCI

Guanidine Carbonate, HCI and Phosphate

Hexamidine Diisethionate and Paraben

Isostearamidopropyl Dimethylamine Gluconate, Glycolate and Lactate

Isostearamidopropyl Morpholine Lactate

Lauryl Isoquinolinium Saccharinate

Lauryl PCA

Lysine DNA and Glutamate

MEA-Benzoate, Dicetearyl Phosphate, o-Phenylphenate, Salicylate, Thiolactate and Undecylenate

MEA-Laureth-6 Carboxylate

MEA PPG-6 Laureth-7 Carboxylate

MEA PPG-8 Steareth-7 Carboxylate

Methyl Hydroxycetyl Glucaminium Lactate

Methylsilanol Hydroxyproline Aspartate

Nicotinyl Tartrate

Olivamidopropyl Dimethylamine Lactate

Oxyquinoline Benzoate and Sulfate

PCA Ethyl Cocoyl Arginate

Piroctone Olamine

Pyridoxine HCI

Saccharated Lime

TEA-Cocoyl Alaninate

TEA-EDTA

TEA-Lauroyl Lactylate

TEA-Phenylbenzimidazole Sulfonate

Thurfylnicotinate HCI

N. Organic Imidazolines

Stearyl Hydroxyethyl Imidazoline

O. Ethoxylated Amines

PEG-15 Tallowamine

PEG-cocopolyamine

P. Quantemized Cellulose

PG-Hydroxyethylcellulose Cocodimonium Chloride

PG-Hydroxyethylcellulose Lauryldimonium Chloride

PG-Hydroxyethylcellulose Stearyldimonium Chloride

Q. Quaternized Silicone

Quaternium-80*

Silicone Quaternium-1* to 13*

R. Multifunctional Quaternaries

Quaternium-77*, 78*, 81*, 82* and 85*

S. Tertiary Substituted Quaternaries

Tricetylmonium Chloride

*The composition of this material is identified in the International Cosmetic Ingredients Dictionary and Handbook, 8th ed. (2000), the Cosmetic Toiletry and Fragrance Association, 1101 17th St., NW, Suite 300, Washington, D.C. 20036-4702.

The cationic polymers that may be compatibilized with anionic polymers according to the invention are especially polymers of the polyamine, polyaminoamide or poly-(quaternary ammonium) type, the amino and/or ammonium group forming part of the polymer chain or being joined thereto. Amongst these polymers, the following may be mentioned in particular:

Vinylpyrrolidone/dialkylaminoalkyl acrylate or methacrylate copolymers (quaternized or unquaterinized), such as those sold under the name Gafquat®, such as “copolymer 845” and Gafquat® 734 or 755, described in greater detail in particular in U.S. Pat. Nos. 3,910,862 and 4,126,537 which are incorporated herein by reference.

Cellulose ether derivatives containing quaternary ammonium groups, such as those described in U.S. Pat. No. 3,472,840, and cationic cellulose derivatives, such as those described in U.S. Pat. No. 4,131,576 which are incorporated herein by reference.

Cationic polysaccharides, such as those described in U.S. Pat. Nos. 3,589,978 and 4,031,357, which are incorporated herein by reference, and in particular Jaguar® C, 13 S sold by Meyhall.

Cationic polymers chosen from the group comprising:

(a) Polymers containing units of the formula:

—A^{4—Z—A}^{4—Z} (I)

in which A⁴ denotes a radical containing two amino groups, preferably a piperazinyl radical, and Z denotes the symbol B or B′; B and B′, which are identical or different, denote a divalent radical which is a straight-chain or branched-chain alkylene radical which contains up to 7 consecutive carbon atoms in the main chain, which is unsubstituted or substituted by hydroxyl groups and which can also contain oxygen, nitrogen and sulphur atoms and 1 to 3 aromatic and/or hetero-cyclic rings, the oxygen, nitrogen and sulphur atoms being present in the form of ether or thioethers, sulphoxide, sulphone, sulphonium, amine, alkylamine, alkenylamine, benzylamine, amine oxide, quaternary ammonium, amide, imide, alcohol, ester and/or urethane groups; these polymers and the process for their preparation are described in French Patent No. 2,162,025.

(b) Polymers containing units of the formula:

—A^{4—Z}_{1—A}^{4—Z}₁ (II)

in which A⁴ denotes a radical containing two amino groups, preferably a piperazinyl radical, and Z₁ denotes the symbol B₁ or B′₁ and denotes the symbol B′₁ at least once; B₁ denotes a divalent radical which is a straight-chain or branched-chain alkylene or hydroxyalkylene radical having up to 7 consecutive carbon atoms in the main chain, and B′₁ is a divalent radical which is a straight-chain or branched-chain alkylene radical which has up to 7 consecutive carbon atoms in the main chain, which is unsubstituted or substituted by one or more hydroxyl radicals and which is interrupted by one or more nitrogen atoms, the nitrogen atom being substituted by an alkyl chain which has from 1 to 4 carbon atoms, and preferably 4 carbon atoms, which is optionally interrupted by an oxygen atom and which optionally contains one or more hydroxyl groups.

The polymers of the formula (II) and the process for their preparation are described in U.S. Pat. No. 4,013,787, which is incorporated by reference.

(c) The alkylation products of the polymers of the formulae (I) and (II) indicated above with alkyl and benzyl halides and lower alkyl tosylates or mesylates, and the oxidation products of the said polymers.

Optionally alkylated, crosslinked polyamino polyamides chosen from the group comprising at least one water-soluble crosslinked polymer obtained by crosslinking a polyaminopolyamide (A) prepared by the polycondensation of an acid compound with a polyamine. The acid compound is chosen from (i) organic dicarboxylic acids, (ii) aliphatic monocarboxylic and dicarboxylic acids with a double bond, (iii) esters of the above-mentioned acids, preferably the esters with lower alkanols having from 1 to 6 carbon atoms, and (iv) mixtures of these compounds. The polyamine is chosen from amongst bis-primary, mono-secondary or bis-secondary polyalkylenepolyamines. Up to 40 mol % of this polyamine can be replaced by a bis-primary amine, preferably ethylenediamine, or by a bis-secondary amine, preferably pipereazine, and up to 20 mol % can be replaced by hexamethylenediamine. The crosslinking is carried out by means of a crosslinking agent (B) chosen from amongst epihalogenohydrins, diepoxides, dianhydrides, unsaturated anhydrides and bis-unsaturated derivatives, suitably in proportions of 0.025 to 0.35 mol of crosslinking agent per amino group of the polyaminopolyamide (A). These polymers and their preparation are described in greater detail in U.S. Pat. No. 4,172,877, which is incorporated by reference.

The alkylation can be carried out, if appropriate with glycidol, ethylene oxide, propylene oxide or acrylamide.

The polyaminopolyamides (A) themselves can also be used according to the invention.

The crosslinked polyaminopolyamides obtained by crosslinking a polyamino-polyamide (A) described above by means of a crosslinking agent chosen from the group comprising:

(I) compounds chosen from the group comprising (1) bis-halogenohydrins, (2) bis-azetidinium compounds, (3) bis-halogenoacyl-diamines and (4) bis-(alkyl halides);

(II) the oligomers obtained by reacting a compound (a) chosen from the group comprising (1) bis-halogenohydrins, (2) bis-azetidinium compounds, (3) bis-halogenoacyl-diamines, (4) bis-(alkyl halides), (5) epihalogenohydrins, (6) diepoxides and (7) bis-unsaturated derivatives, with a compound (b) which is a difunctional compound reactive towards the compound (a); and

(III) the quatemization product of a compound chosen from the group comprising the compounds (a) and the oligomers (II) and containing one or more tertiary amine groups which can be totally or partially alkylated, with an alkylating agent (c) preferably chosen from the group comprising methyl or ethyl chlorides, bromides, iodides, sulphates, mesylates and tosylates, benzyl chloride or bromide, ethylene oxide, propylene oxide and glycidol, the cross-linking being carried out by means of, say, 0.025 to 0.35 mol, in particular by means of 0.025 to 0.2 mol and more particularly by means of 0.025 to 0.1 mol, of crosslinking agent per amine group of the polyaminopolyamide.

These crosslinking agents and these polymers, and also the process for their preparation, are described in U.S. Pat. No. 4,172,887, which is incorporated herein by reference.

The polyaminopolyamide derivatives resulting from the condensation of a poly-alkylenepolyamine with a polycarboxylic acid, followed by alkylation by means of difunctional agents, such as adipic acid/dialkylaminohydroxyalkyl-dialkylenetriamine copolymers in which the alkyl radical contains 1 to 4 carbon atoms and preferably denotes methyl, ethyl or propyl, which are described in U.S. Pat. No. 3,632,559, which is incorporated herein by reference.

Amongst these compounds, there may be mentioned especially the adipic acid/dimethylaminohydroxypropyl diethylenetriamine copolymers.

The polymers obtained by reacting a polyalkylenepolyamine containing two primary amine groups and at least one secondary amine group with a dicarboxylic acid chosen from amongst diglycolic acid and saturated aliphatic dicarboxylic acids having 3 to 8 carbon atoms, the molar ratio of the polyalkylenepolyamine to the dicarboxylic acid being from 0.8:1 to 1.4:1, and the resulting polyaminoamide being reacted with epichlorohydrin in a molar ratio of epichlorohydrin to the second amine groups of the polyaminoamide of 0.5:1 to 1.8:1; these polymers are mentioned in U.S. Pat. Nos. 3,227,615 and 2,961,347, which are incorporated herein by reference.

Cyclic polymers generally having a molecular weight of 20,000 to 3,000,000, such as homopolymers containing, as the main constituent of the chain, units corresponding to the formula (III) or (III′):

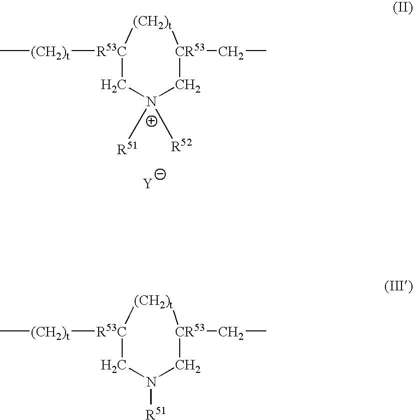

in which 1 and t are equal to 0 or 1 with 1+t=1, R⁵³ denotes hydrogen or methyl, R⁵¹ and R⁵² independently of one another denote an alkyl group having from 1 to 22 carbon atoms, a hydroxyalkyl group in which the alkyl group preferably has 1 to 5 carbon atoms, or a lower amidoalkyl group, and R⁵¹ and R⁵² can denote, together with the nitrogen atom to which they are attached, heterocyclic groups such as a piperidinyl or morpholinyl; Y⊖ is an anion such as bromide, chloride, acetate, borate, citrate, tartrate, bisulphate, bisulphate, sulphate or phosphate and also copolymers containing units of the formulas III or III′ and units derived from acrylamide or from diacetone-acrylamide.

Amongst the quaternary ammonium polymers of the type defined above, there may be mentioned the dimethyldiallylammonium chloride homopolymer sold under the name MERQUAT 100 and having a molecular weight of less than 100,000, and the dimethyl-diallylammonium chloride/acrylamide copolymer having a molecular weight of more than 500,000 and sold under the name MERQUAT 550.

These polymers are described in U.S. Pat. No. 3,986,825, which is incorporated herein by reference.

Poly-(quaternary ammonium) compounds of the formula:

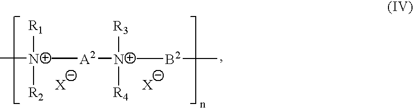

in which R₁ and R₂, and R₃ and R₄, which are identical or different, represent aliphatic, alicyclic or arylaliphatic radicals containing at most 20 carbon atoms, or lower hydroxyaliphatic radicals, or alternative R₁ and R₂, and R₃ and R₄, together or separately form, with the nitrogen atoms to which they are attached, heterocyclic rings optionally containing a second hetero-atom other than nitrogen, or alternatively R₁, R₂, R₃ and R₄ represent a group

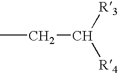

R′₃ denoting hydrogen or lower alkyl and R′₄ denoting

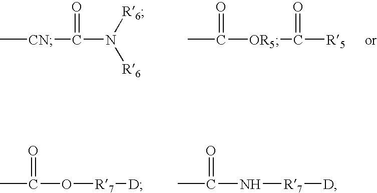

R′₅ denoting lower alkyl, R′₆ denoting hydrogen or lower alkyl, R′₇ denoting alkylene and D denoting a quaternary ammonium group; A² and B² can represent polymethylene groups containing from 2 to 20 carbon atoms, which can be linear or branched and saturated or unsaturated and which can contain, inserted into the main chain, one or more aromatic rings such as the group

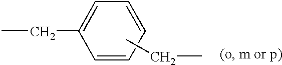

or one or more groups —(CH₂)ₙ—Y—(CH₂)ₙ—, Y denoting O, S, SO, SO₂,

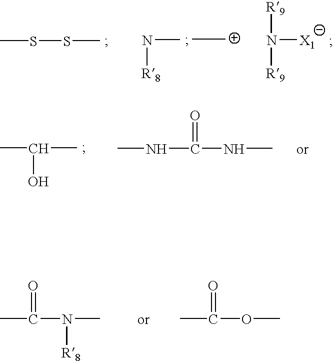

with X₁^{⊖} denoting an anion derived from a mineral or organic acid, n denoting 2 or 3, R′8 denoting hydrogen or lower alkyl and R′9 denoting lower alkyl, or alternatively A² and R1 and R3 form a piperazine ring with the two nitrogen atoms to which they are attached; moreover, if A² denotes a linear or branched, saturated or unsaturated alkylene or hydroxyalkylene radical, B² can also denote a group—(CH₂—)ₙC(O)—D—(O)C—(CH₂)ₙ, in which D denotes:

(a) a glycol radical of the formula —O—Z′—O—, in which Z′ denotes a linear or branched hydrocarbon radical or a group corresponding to the formulae:

—[CH₂—CH₂—O]—ₓCH₂—CH₂—

or

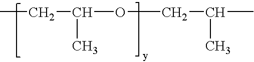

in which x and y denote an integer from 1 to 4, representing a definite and unique degree of polymerization (in the case of a single compound), or any number from 1 to 4, representing an average degree of polymerization (in the case of a mixture);

(b) a bis-secondary diamine radical, such as a piperazine derivative;

(c) a bis-primary diamine radical of the formula: —N—H—Y—NH—, in which Y denotes a linear or branched hydrocarbon radical or the divalent radical —CH₂—CH₂—S—S—CH₂—CH₂—; or

(d) a ureylene group of the formula —N—H—CO—NH—; n is such that the molecular weight is generally from 1,000 to 100,000 and X^{⊖} denotes an anion.

Polymers of this type are described in particular in U.S. Pat. Nos. 4,075,136; 4,217,914;1 4,197,865; 4,349,532; 2,273,780; 2,375,853; 2,388,614; 2,454,547; 3,206,462; 2,261,002 and 2,271,378, which are incorporated herein by reference..

Other polymers of this type are described in U.S. Pat. Nos. 3,874,870, 4,001,432, 3,929,990, 3,966,904, 4,005,193, 4,025,617, 4,025,627, 4,025,653, 4,026,945 and 4,027,020.

Homopolymers or copolymers derived from acrylic or methacrylic acid and containing at least one unit:

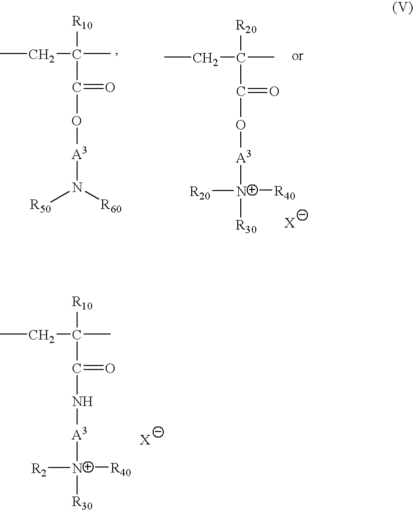

in which R₁₀ is H or CH₃, A³ is a linear or branched alkyl group having 1 to 6 carbon atoms or a hydroxyalkyl group having 1 to 4 carbon atoms, R₂₀, R₃₀ and R₄₀, which are identical or different, denote an alkyl group having 1 to 18 carbon atoms or a benzyl group, R₅₀ and R₆₀ denote H or alkyl having 1 to 6 carbon atoms, and X denotes methosulphate or halogen, such as chlorine or bromine.

The comonomer or comonomers which can be used include: acrylamide, metha-crylamide, diacetoneacrylamide, acrylamide and methacrylamide substituted on the nitrogen by one or more lower alkyls, acrylic and methacrylic acid esters, vinylpyrrolidone and vinyl esters.

The following may be mentioned by way of example: the acrylamide/beta-methacryloyloxethyl-trimethylammonium methosulphate copolymers, the aminoethylacrylate phosphate/acrylate copolymer, and the graft crosslinked cationic copolymers, having a molecular weight of 10,000 to 1,000,000 and preferably of 15,000 to 500,000. These polymers are described in U.S. Pat. No. 3,946,749, which is incorporated herein by reference.

The cosmetic monomer can be of a very wide variety of types, for example a vinyl ester, an allyl or methallyl ester, an acrylate or methacrylate of a saturated alcohol having from 1 to 18 carbon atoms, an alkyl vinyl ether, an olefin, a vinylic heterocyclic derivative, a dialkyl or N,N-dialkylaminoalkyl maleate or an unsaturated acid anhydride.

Quaternary polymers of vinylpyrrolidone and vinylimidazole, such as LUVIQUAT FC 905.

Cationic silicone polymers, such as those described in European Patent Nos. 0017121 B1 and 0017122 B1 and U.S. Pat. No. 4,185,087, which are incorporated herein by reference.

Cationic derivatives of starches or of starch ethers, such as those described in Great Britain Patent No. 2063282 which is incorporated herein by reference.

Other cationic polymers which can be used include polyalkyleneimines, in particular polyethyleneimines, polymers containing vinylpyridinium units in the chain, polyamine/epichlorohydrin condensates, poly-(quaternary ureylene) compounds and chitin derivatives.

Compatibilizing Agents

The compatibilizing agents or complexing agents which complex with the cationic materials may be any material that contains a “bulky” molecule having an anionic group. The “bulky” molecule should not be reactive chemically with either the anionic polymer or the cationic material. The “bulky” molecule will generally have a molecular weight of at leasat 500 Mn, preferably at least 1,000 Mn, and may have a molecular weight of up to 50,000 Mn, but generally up to 25,000 Mn. Usually the “bulky” molecule is a polymeric material having at least three repeat units. The composition of the polymeric materials may be heterogeneous and predominantly may be polysilicones, acrylic copolymers, polyalkylene glycol such as polyethylene glycol and polypropylene glycol, polyvinyl alcohol, polyvinyl acetate, polysaccharide such as starch and cellulose or polyurethane. Polyalkylene glycols may contain terminal groups such as, but not limited to, allyl, propenyl, propyl and hydrogen or others. These polymeric or “bulky” groups must contain anionic groups that will complex with the cationic materials. The preferred anionic groups are carboxylate (—COOH), sulfonate (—SO₃H), sulfate (—OSO₃H), phosphate (—OP(OH)₂) and phosphonate (—PO(OH)₂). The anionic groups complex with the cationic materials preventing the cationic materials from interfering with the anionic polymer and permitting the anionic polymer to perform its function, such as the viscosity building function in the case of thickeners or a film forming function in the case of a coating. Although, in principle, any polymeric material containing anionic groups may be employed, it is preferable to employ silicones, especially in personal care products.

As mentioned above, the compatibilizing agent can be any material that contains a “bulky” molecule having an anionic group, but the complexing agent must be compatible with the cationic material. By “compatible” is meant that the complexing agent and the cationic material do not form a precipitate. Consequently, it is possible that a particular complexing agent may be compatible with certain category or categories of cationic materials but incompatible with different cationic materials. It is also possible that a particular anionic polymer may be compatible with a particular type of cationic material and, therefore, can function as a complexing agent in compatibilizing such cationic material with anionic polymers which are not compatible with such cationic materials. In other words, certain anionic polymers may function as complexing agents, but with respect to other cationic materials, with which those certain anionic polymers are not compatible, different complexing agents must be employed to compatibilized those cationic materials with those certain anionic polymers.

The preferred silicone complexing agents may be represented generically

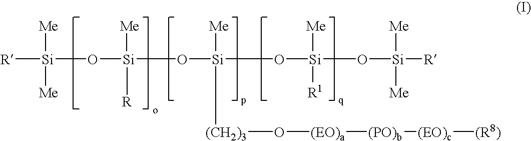

wherein:

Me is methyl;

R and R′ are independently selected from methyl, —OH, —R⁷, and —R⁹—A or —(CH₂)₃—O—(EO)ₐ—(PO)_{b}—(EO)_{c}—G with the proviso that both R and R′ are not methyl, —OH or R⁷;

R¹ is selected from lower alkyl CH₃(CH₂)ₙ— or phenyl where n is an integer from 0 to 22;

a, b, and c are integers independently ranging from 0 to 100;

EO is —(CH₂CH₂O )—;

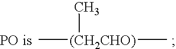

o is an integer ranging from 1 to 200;

q is an integer ranging from 0 to 1000;

p is an integer ranging from 0 to 200;

R⁷ is aryl, alkyl, aralkyl, alkaryl, or alkenyl group of 1-40 carbons;

R⁸ is hydrogen or R⁷ or C(O)—X wherein X is aryl, alkyl, aralkyl, alkaryl, alkenyl group of 1-40 carbons, or a mixture thereof,

R⁹ is divalent group selected from alkylene of 1-40 carbons which may be interrupted with arylene group of 6 to 18 carbons or an alkylene group containing unsaturation of 2 to 8 carbons;

A and G are independently are selected from

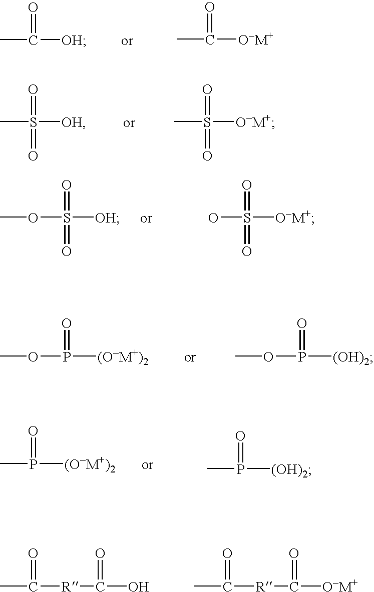

where

R″ is a divalent group selected from alkylene of 1-40 carbons which may be interrupted with an arylene group of 6 to 18 carbons or an alkylene group of 2 to 8 carbons, and is preferably selected from the

R″ is selected from —CH₂—CH₂—; —CH═CH—;

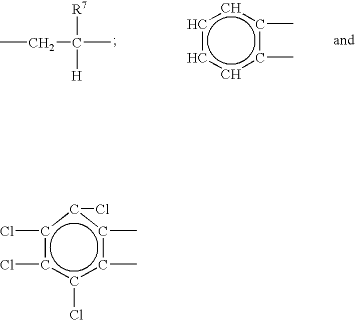

where M is Na, K, Li, NH₄; or an amine containing alkyl, aryl, akenyl, hydroxyalkyl, arylalkyl or alkaryl groups.

Another category of silicone complexing agents is silicone sulfates which may be represented by the following formula:

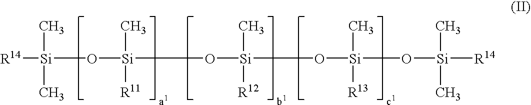

wherein

R¹¹ is selected from lower alkyl having one to eight carbon atoms or phenyl,

R¹² is —(CH₂)₃—O—(EO)ₓ—(PO)_{y}—(EO)_{z}—SO₃³¹M⁺

M is a cation and is selected from Na, K, Li, or NH₄;

x, y and z are integers independently ranging from 0 to 100;

R¹³ is —(CH₂)₃—O—(EO)ₓ—(PO)_{y}—(EO)_{z}—H

R¹⁴ is methyl or hydroxyl;

a¹ and c¹ are independently integers ranging from 0 to 50;

b¹ is an integer ranging from 1 to 50;

A still further category of silicone complexing agents may be represented as follows:

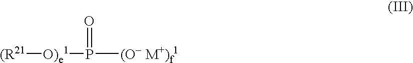

wherein

R²¹ is

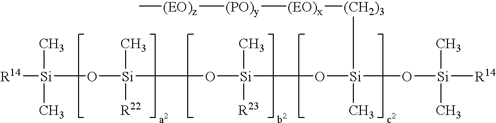

a² is an integer from 0 to 200;

b² is an integer from 0 to 200;

c² is an integer from 1 to 200;

R¹⁴ is as defined above;

R²² is selected from —(CH₂)ₙCH₃ and phenyl;

n is an integer from 0 to 10;

R²³ is —(CH₂)₃—O—(EO)ₓ₁—(PO)_{y1}—(EO)_{z1}—H;

x¹, y¹ ands z¹ are integers and are independently selected from 0 to 20;

e¹ and f¹ are 1 or 2 with the proviso that e+f=3;

M is selected from H, Na, K, Li, or NH₄; and

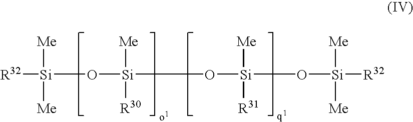

wherein;

Me is methyl;

R³⁰ and R³² independently are CH₃ or —(CH₂)₃—O—(EO)ₐ_{³}—(PO)_{b}_{³}—(EO)_{c}_{³}—C(O)—R³³—C(O)—OH;

with the proviso that both R³⁰ and R³² are not —CH₃;

R³³ is selected from —CH₂—CH₂—; —CH═CH—; —CH₂—C(R³⁷)—H;

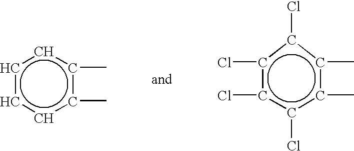

R³⁷ is alkyl having from 1 to 22 carbon atoms;

R³¹ is selected from lower alkyl (having 1-4 carbons), CH₃(CH)ₙ¹— and phenyl;

n¹ is an integer from 0 to 8;

a³, b³ and c³ are integers independently ranging from 0 to 20;

EO is an ethylene oxide residue —(CH₂CH₂—O)—;

PO is a propylene oxide residue —(CH₂CH(CH₃)—O—);

o¹ is an integer ranging from 1 to 200;

q¹ is an integer ranging from 0 to 500.

It should be noted that in the above structure units EO and PO may be in random and block structures.

Such silicone carboxylates are disclosed in greater detail in U.S. Pat. No. 5,296,625, the disclosure of which is incorporated herein by reference. Still further silicone complexing agents are silicones containing a multiplicity of different anionic substituents. Such silicones can be prepared by reacting two or more types of anionic silicones already disclosed using reactions well known to those in the art. The resulting molecule could be a hybrid of the starting silicones and would, therefore, contain multiple types of anionic functional groups. The properties of the silicone can be optimized in such a fashion. One type of reaction, the silicone equilibration reaction, involves charging a reactor with raw materials, adding a suitable catalyst, mixing with heat, and then neutralizing the catalyst. The Chemistry is discussed in Silicone in Organic, Organometallic and Polymer Chemistry (Michael Brook)—John Wiley and Sons, New York, 2000, pp. 261-266.

The amount of the anionic complexing agent required to complex the cationic materials will depend on the specific cationic materials (the quat, polyquat, organic salt, etc.), the amount of the cationic materials present and the overall pH of the final formulation. The lower the pH of the final formulation, the greater the amount of the complexing agent is required. In view of the above-mentioned variables, it will be necessary to conduct some routine testing to arrive at the optimum amount of the anionic complexing agent, such as a silicone, to be used in a particular formulation to provide the desired results. Generally, the weight ratio of the anionic complexing agent, such as the anionic silicone complexing agent, to the cationic material or materials, will be in the range of 0.1-10.1. Preferably, the weight ratio of the complexing agent to the cationic ingredient(s) will be 0.5-6 to 1 and most preferably 1.5-3 to 1.

Anionic Polymers

The anionic polymers which are generally not compatible with cationic materials, can be homopolymers obtained from ethylenically unsaturated monomers containing acid moieties, such as carboxyl, phosphate, phosphonate, sulfate, sulfonate, phenolic, or any other moiety having a labile hydrogen that can be removed from the moiety to provide a negatively charged site on the polymer or ethylenically unsaturated monomers derived from those that contain carboxylic groups, such as acid hydrides, anhydrides or esters. The anionic polymers may also be copolymers of ethylenically unsaturated monomers containing the above-noted acid moieties and one or more monomer(s) that are copolymerizable with the acid group-containing monomers. Such copolymers will contain at least 1% by weight of one or more acid groups containing monomer(s) or anhydride monomer(s), preferably at least 5% and more preferably at least 10% and often at least 25%. Prior art discloses a variety of such homopolymers and copolymers, some of which may be rheology modifiers (thickeners) and others may be non-thickeners that do not substantially and effectively modify rheology of a composition to which an anionic polymer has been added.

It may be noted that the general chemical compositions of non-thickener polymers are very similar, or in some cases identical, to those of thickener polymers. The more notable difference between the two types of polymers are performance-related. Although chemical differences exist, they are subtle and not always evident. An adjustment in polymer architecture or charge density, a small change in composition, a slight increase or decrease in the amount of a certain monomer or crosslinker can all make the difference between a thickener and a non-thickener.

A thickener is a material or an ingredient in a formulation whose function is to provide a viscosity increase, stabilize suspended ingredients such as solid particles or emulsion droplets, and/or to otherwise modify the rheology of the formulation. Rheology modifiers are well known in the art and can be prepared from synthetic and/or natural polymers. Thickening may be the primary function of the ingredient, or a secondary effect. Thickeners are usually used at <10 weight percent in a formulation, and more preferably <5%. Some very efficient thickeners are used at <1% levels.

The broad category of anionic polymers includes anionic rheology modifiers (thickeners). This category of polymers, that includes homopolymers and copolymers, has been disclosed in the parent patent application, U.S. Ser. No. 10/653,609, filed Sep. 2, 2003, which disclosure is incorporated herein by reference. The present application deals with compatibilizing cationic materials with anionic polymers that are preferably non-thickeners, that is, polymers that have useful properties other than as rheology modifiers.

Carboxylic monomers are an important category of acid moiety-containing monomers from which anionic homopolymers and copolymers, other than thickeners, may be prepared. By “non-thickener” is meant that such polymers may possess no thickener or rheology modifying property or that the polymers may exhibit some thickening property but not sufficient to be categorized as a commercially viable rheology modifier. Useful in the production of such non-thickener anionic polymers are the olefinically unsaturated carboxylic acids containing at least one activated carbon-to-carbon olefinic double bond, and at least one carboxyl group, that is, an acid containing an olefinic double bond which readily polymerizes because of its presence in the monomer molecule either in the alpha-beta position with respect to a carboxyl group,

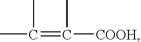

or as a part of a teminal methylene grouping thusly, CH₂═C<. In the alpha-beta acids, the close proximity of the strongly polar carboxyl group to the double-bonded carbon atoms has a strong activating influence rendering the substances containing this structure very readily polymerizable. The presence of a terminal methylene grouping in a carboxylic monomer makes this type of compound much more easily polymerizable than if the double bond were intermediate in the carbon structure. Olefinically-unsaturated acids of this class include such widely divergent materials as the acrylic acids typified by acrylic acid itself, methacrylic acid, ethacrylic acid, alpha-chloroacrylic acid, alpha-cyano acrylic acid, beta methyl-acrylic acid (crotonic acid), alpha-phenyl acrylic acid, beta-acryloxy propionic acid, sorbic acid, alpha-chloro sorbic acid, angelic acid, cinnamic acid, p-chloro cinnamic acid, beta-styryl acrylic acid (1-carboxy-4-phenyl butadiene-1,3), itaconic acid, citraconic acid, messaconic acid, glutaconic acid, aconitic acid, maleic acid, fumaric acid, and tricarboxy ethylene. As used herein, the term “carboxylic acid” includes the polycarboxylic acids and those acid anhydrides, such as maleic anhydride, wherein the anhydride group is formed by the elimination of one molecule of water from two carboxyl groups located on the same polycarboxylic acid molecule. Anhydrides of the types formed by elimination of water from two or more molecules of the same or different unsaturated acids, such as acrylic anhydride, are not included because of the strong tendency of their polymers to hydrolyze in water and alkali. Maleic anhydride and the other acid anhydrides useful herein have the general structure:

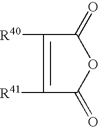

wherein R⁴⁰ and R⁴¹ are independently selected from the group consisting of hydrogen, cyanogens (—C═N), hydroxyl, lactam and lactone groups and alkyl, aryl, alkaryl, aralkyl, and cycloalkyl groups such as methyl, ethyl, propyl, octyl, decyl, phenyl, tolyl, xylyl, benzyl, cyclohexyl and the like.

The preferred carboxylic monomers for use in preparing non-thickener anionic polymers are the monoolefinic acrylic acids having the general structure

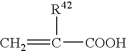

wherein R⁴² is a substituent selected from the class consisting of hydrogen, halogen, hydroxyl, lactone, lactam cyanogen (—CN), monovalent alkyl group (1 to 4 carbons), monovalent aryl group (6 to 12 carbons), monovalent aralkyl group (7 to 12 carbons), monovalent alkaryl group (7 to 12 carbons) and monovalent cycloaliphatic group (4 to 8 carbons). Of this class, acrylic acid and methacrylic acid are most preferred because of its generally lower cost, ready availability, and ability to form superior polymers. Another particularly preferred carboxylic monomer is maleic anhydride.

In addition to the carboxyl group-containing monomers, other ethylenically unsaturated acid group-containing monomers may be vinyl sulfonic acids, vinylsulfuric acid, vinylphosphonic acids, vinylaromatic acids wherein the acid groups are carboxylic, sulfonic, sulfuric or phosphonic, and even phenolic; that is, any moiety having a labile hydrogen that can be removed from the moiety to provide a negatively charged site on the polymer. Illustrative examples of such acidic monomers are vinylsulfonic acid, styrenesulfonic acid, 2-methacroyloxyethane-1-sulfonic acid, 3-(vinyloxy) propane-1-sulfonic acid, 3-methacryloxypropane-1-sulfonic acid, vinylsulfuric acid, vinylphos-phine acid, 4-vinylbenzoic acid, 4-vinylphenol, 4-vinylphenyl sulfuric acid, N-vinylsuccinamidic acid and other similar acids. An anionic polymer containing an aromatic monomer can be sulfonated, sulfated or phosphonated to position an acid group on the aromatic monomer.

The above-discussed acid group-containing monomers may be homopolymerized or copolymerized with a variety of other monomers, especially vinyl type. An important group of comonomers are acrylic ester monomers having long chain aliphatic groups which are derivatives of acrylic acid represented by the formula

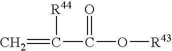

wherein R⁴³ is hydrogen or an alkyl group having from 1 to 30 carbon atoms, preferably 10 to 22 carbon atoms and R⁴⁴ is hydrogen or a methyl group. Representative acrylates include methyl acrylate, ethyl acrylate, propyl acrylate, isopropyl acrylate, butyl acrylate, isobutyl acrylate, methyl methacrylate, methyl ethacrylate, ethyl methacrylate, octyl acrylate, heptyl acrylate, octyl methacrylate, isopropyl methacrylate, 2-ethylhexyl acrylate, nonyl acrylate, hexyl acrylate, n-hexyl methacrylate, and the like; higher alkyl acrylic esters are decyl acrylate, isodecyl methacrylate, lauryl acrylate, stearyl acrylate, behenyl acrylate and melissyl acrylate and the corresponding methacrylates. Mixtures of two or three or more long chain acrylic esters may be successfully polymerized with one of the carboxylic monomers.

Other vinylidene monomers may also be used, including the acrylic nitriles, α,β-olefinically unsaturated nitriles useful are preferably the monoolefinically unsaturated nitriles having from 3 to 10 carbon atoms such as acrylonitrile, methacrylonitrile, ethacrylonitrile, chloroacrylonitrile, and the like. Acrylic amides include monoolefinically unsaturated amides also may be used. These have at least one hydrogen on the amide nitrogen and the olefinic unsaturation is alpha-beta to the carbonyl group. Representative amides include acrylamide, methacrylamide, N-methylacrylamide, N-t-butyl acrylamide, N-cyclohexyl acrylamide, N-ethyl acrylamide and others. Other N-alkylol amides of alpha, betaolefinically unsaturated carboxylic acids including those having from 4 to 10 carbon atoms such as N-methylol acrylamide, N-ethanol acrylamide, N-propanol acrylamide, N-methylol methacrylamide, N-ethanol methacrylamide, N-methylol maleimide, N-methylol maleamide, N-methylol maleamic acid, N-methylol maleamic acid esters, the N-alkylol amides of the vinyl aromatic acids such as N-methylol-p-vinyl benzamide, and the like and others. N-alkoxymethyl acrylamides also may be used.

Other useful vinylidene comonomers include α-olefins containing from 2 to 12 carbon atoms, dienes containing from 4 to 10 carbon atoms; vinyl esters and allyl esters such as vinyl acetate; vinyl aromatics such as styrene, methylstyrene, cholorstyrene, vinyl and allyl ethers and ketones such as vinyl methyl ether and methyl vinyl ketone; chloroacrylates, cyanoalkyl acrylates such as α-cyanomethyl acrylate, the α-, β and γ-cyanopropyl acrylate, alkoxyacrylates such as methoxy ethyl acrylate; haloacrylates as chloroethyl acrylate, vinyl halides and vinyl chloride, vinylidene chloride and the like; vinyl benzyl chlorides; esters of maleic and fumaric acid and the like; divinyls, diacrylates and other polyfunctional monomers such as divinyl ether, diethylene glycol diacrylate, ethylene glycol dimethacrylate, methylene-bis-acrylamide, allylpentaerythritol, and the like; and bis(β-chloroethyl) vinyl phosphonate and the like as are known to those skilled in the art. These copolymers are disclosed in greater detail in U.S. Pat. No. 4,419,502 which is incorporated herein by reference. Also useful comonomers are alkoxyalkyl acrylates, methacrylates or esters of the other above-metioined unsasturated carboxylic acids wherein the alkyl groups have 1 to 18 carbons.

Another class of comonomers that may be employed in preparing anionic polymers is associative monomers which is an ester of formula

J—O—(CH₂—CHR₂₂O)ᵣ—(CH₂)ₛ—R₁₁

wherein

J is an ethylenically unsaturated acrylic residue, optionally containing an additional carboxylic group, wherein, optionally, said additional carboxylic group may be esterified with a (C₁-C₂₀) aliphatic alkyl group;

R₁₁ is an alkyl, alkylphenyl or aralkyl residue having from 1 to 30 carbon atoms;

R₂₂ is hydrogen or alkyl of 1 to4 carbons;

r is comprised between 0 and 50;

s is comprised between 0 and 30.

The associative monomer may be any compound falling within the above formula J—O—(CH₂—CHR₂₂O)ᵣ—(CH₂)ₛ—R₁ wherein R₁₁ and R₂₂ are as above indicated, the sum of r and s may vary between 0 and 80 and J is the acrylic residue of an ethylenically unsaturated acid selected from acrylic, methacrylic, itaconic, maleic, sorbic, crotonic, oleic and linoleic acids. Preferred are the esters of cetylstearyl alcohol ethoxylated with 25 moles of ethylene oxide. The associative monomers are commercially available products, or they can be prepared substantially according to procedures known in the art (U.S. Pat. Nos. 3,652,497 and 4,075,411).

Crosslinking monomers may be employed in anionic polymers, if desired. If one is employed, a preferred class is polyalkenyl polyether having more than one alkenyl ether grouping per molecule. The most useful possess alkenyl groups in which an olefinic double bond is present attached to a terminal methylene grouping, CH₂═C<. They are made by the etherification of a polyhydric alcohol containing at least 4 carbon atoms and at least 3 hydroxyl groups. Compounds of this class may be produced by reacting an alkenyl halide, such as allyl chloride or allyl bromide with a strongly alkaline aqueous solution of one or more polyhydric alcohols. The product is a complex mixture of polyethers with varying numbers of ether groups. Analysis reveals only the average number of ether groupings on each molecule. Efficiency of the polyether crosslinking agent increases with the number of potentially polymerizable groups on the molecule. It is preferred to utilize polyethers containing an average of two or more alkenyl ether groupings per molecule. Other crosslinking monomers include for example, diallyl esters, dimethallyl ethers, allyl or menthally acrylates and acrylamides, tetraallyl tin, tetravinyl silane, polyalkenyl methanes, diacrylates and dimethacrylates, divinyl compounds, polyallyl phosphate, diallyloxy compounds and phosphite esters and the like. A more complete listing of crosslinkers may be found in U.S. Pat. Nos. 4,190,562; 3,639,459 and 4,138,381 which are incorporated herein by reference.

Anionic polymers, including thickening agents, are available commercially from many suppliers under a variety of trade names. Thus, Noveon, Inc. (formerly The B.F. Goodrich Company) sells a variety of various acrylate copolymers containing an unsaturated carboxylic acid, usually acrylic acid and a variety of esters of acrylic or methacrylic acid, under the name of AVALURE AC; BASF sells under the names of LUVIMER MAE and LUVIFLEX. Noveon, Inc. also sells CARBOSET resins that are copolymers of unsaturated carboxylic acids having 1 to 3 carboxyl groups and acrylates or methacrylates having 1-18 carbon alkyl groups, including stearyl and GOOD-RITE resins which are copolymers containing acrylamidomethyl propane sulfonic acid or vinylphosphonic acid or itaconic acid. Anionic copolymers are also available from Rohm & Haas as ACRYSOL, KARAMUL, FRANCONYX and PRIMAL which are aqueous acrylic emulsions.

Other anionic polymers are copolymerse of (meth)acrylic acid, of linear or branched C₁-C₂₀ alkyl (meth)acrylate and of vinylpyrrolidone, such as that sold by the company ISP under the name ACRYLIDONE LM;

(meth)acrylic acid/alkyl acrylate/alkyl methacrylate copolymers, in particular the ethyl acrylate/methyl methacrylate/methacrylic acid/acrylic acid copolymer, such as the product sold under the name AMERHOLD DR 25 by the company Amerchol;

copolymers of (meth)acrylic acid and of at least one acrylamide which are optionally N-substituted, sold for example under the names RETEN 421, 423 or 425 by the company Hercules or under the names ULTRAHOLD by the company BASF;

copolymers of (meth)acrylic acid and of styrene; and

copolymers of (meth)acrylic acid and of vinylpyrrolidone such as that sold under the name ACRYLIDONE ACP 1001 by the company ISP.

Still further anionic polymers are the acrylic acid/ethyl acrylate/N-tertbutylacrylamide copolymers sold under the name ULTRAHOLD STRONG by the company BASF and the copolymers of methacrylic acid and of methyl methacrylate sold under the name EUDRAGIT by the company Rohm Pharma.

Also available are vinyl acetate/crotonic acid copolymer LUVISET CA-66 and acrylates/dimethicone copolymer as LUVIFLEX SILK, both from BASF. Polymethacrylic acid/acrylamidomethyl propane sulfonic acid copolymer is sold by Ondea/Nalco as FIXOMER A-30 and various monoalkyl esters of ethyl, isopropyl and butyl of poly(methyl vinyl ether/maleic acid) are sold as GANTREZ copolymers by ISP which also sells acrylates/hydroxyester acrylate copolymer as ACUDYNE; polyvinyl pyrrolidone/acrylates/lauryl methacrylate copolymer as STYLEZE 2000; vinyl acetate/butyl maleate/isobomyl acrylate copolymer under the name of ADVANTAGE; polydimethylsiloxane encapsulated in polyvinyl-pyrrolidone or polyvinyl pyrrolidone copolymer as GAFQUAT HSi and quaternized copolymer of vinyl pyrrolidoine and dimethylaminoethyl methacrylate as GAFQUAT 755NP or 440.

National Starch markets RESYN products which are vinyl acetate/crotonic acid copolymer or vinyl acetate/crotionic acid/vinyl neodecanoate copolymer.

Many anionic polymers as rheology modifiers or thickeners are also commercially available from various suppliers. Noveon, Inc. sells Carbopol® thickener resins in a variety of grades and products for various uses and applications. 3V/Sigma supplies a series of thickener products under the Synthalen® series, Stabylen®, PNC® and Polygel®. Rita sells the Acritamer® series of products. Pomponesco sells Addensante®, Gelacril® and Polacril® polymers. BASF sells Luvigel® and Sumitomo Seika sells Aqupec®. The following companies market their corresponding thickener polymers: Goldschmidt AG—TX®; Nihon—Junlan®; Clariant—Aristoflex®; Alban Muller International—Amigel®; Corel Pharma Chem—Acrypol®; Elementis—Rheolate®; Wako Pure Chemical Ind.—Hiviswako®; Rhome & Haas—Aculyn® series; Ciba Specialty Chemicals—Salcare® series; ISP—Stabileze® series; National Starch and Chemical—Structure®series; and Seppic—Capigel® series, Sepigel® series and Simulgel® series.

Neutralizing Agents

In formulations containing anionic polymers, it is often necessary to neutralize the carboxylic moiety. Neutralization is accomplished with one or more inorganic bases such as sodium hydroxide, potassium hydroxide, ammonium hydroxide and/or ammonium carbonate. Useful neutralizing organic bases are primary, secondary and tertiary amines and the water soluble alkanol amines such as monoethanolamine (MEA), diethanolamine (DEA), triethanolamine (TEA), 2-methyl-2-amino-1-propanol (AMP), 2-amino-2-methyl-propanol and 2-amino-2-methyl-1,3-propanediol, respectively, 2-dimethylaminoethanol N,N-dimethyl-ethanolamine), 3-dimethylamino-1-propanol, 3-dimethylamino-2-propanol, 1-amino-2-propanol, and the like, monoamino glycols, and the like, which help solubilize the polymer in water solutions. The level of neutralization required varies for each polymer. The block copolymers become soluble in water and hydroalcoholic solutions at 20% to 100% neutralization. The pH of these solutions usually ranges from 4 to 12 but generally will be between 5 and 8. The lowest neutralization level needed to render the polymer water soluble or dispersible depends on the composition of the polymer and other materials

Other Additives

Most end-use products, whether coatings, household products or personal care products, may benefit from the use of complexed cationic materials of this invention if anionic polymers are also employed in such products. The end-use products usually will also contain additional additives to enhance the desired properties. Such additional additives may include non-volatile silicone compound(s) or other conditioning agent(s), preferably a water-insoluble, emulsifiable conditioning agent, such as a polydimethylsiloxane compound, “silicone gums” which are the non-functional siloxanes having a viscosity of from about 5 to about 600,000 centistokes at 25° C.; rigid silicones; “dimethicone copolyols” which may be inear or branched that may be block or random copolymers; volatile hydrocarbon, such as a hydrocarbon including from about 10 to about 30 carbon atoms.

Another additive that may be incorporated is a soluble tension reducing compound, such as dimethiconecopolyols, panthenol, fluorosurfactants, glycerin POE, PPG 28 Buteth 35, PEG 75 lanolin, oxtoxynol-9, PEG-25 hydrogenated castol oil, polyethylene glycol 25 glyceryl trioleate, oleth-3 phosphate, PPG-5-ceteth-10 phosphate, PEG-20 methyl glucose ether, or glycereth-7-triacetate, glycereth-7 benzoate or combinations thereof.

Also useful additives are plasticizing compounds, such as polycarboxylic acid esters that have a carbon backbone of from 3 to 12 carbon atoms and 3 or more C₁-C₅ alkyl carboxylate groups attached thereto. Illustrative examples are triethyl citrate, tributyl citrate, triethyl phthalate, tributyl phthalate, tripentyl phthalate or combinations thereof. The polycarboxylic add esters are selected from triethyl citrate, tributyl citrate, tributyl phthalate, or combinations thereof.

Possible further useful additives are:

UV Absorbers like butyloctyl salicylate, octylmethoxycinnamate, avobenzone, benzophenone-3 and benzophenone-4, octyl salicylate, para-aminobenzoric acid (PABA), octyldimethyl PABA, hindered cyclic amine UV-light stabilizers based on 3.5-hindered piperidines available as TINUVIN® series of products from Ciba Specialty Chemicals or 3.5-hindered-2-keto-piperazinones.

Surfactants like alcohols, alcohol ethoxylates, alkanolamine-derived amides, ethoxylated amides, amine oxides, ethoxylated carboxylic acids, ethoxylated glycerides, glycol esters and derivatives thereof, monoglycerides, polyglyceryl esters, polyhydric alcohol esters and ethers, sorbitan/sorbitol esters, trimesters of phosphoric acid, ethoxylated lanolin, silicone polyethers, PPO/PEO ethers, alkylpolyglycosides, acyl/dialkyl ethylenediamines and derivatives, n-alkyl amino acids, acyl glutamates, acyl peptides, sarcosinates, taurates, alkanoic acids, carboxylic acid esters, carboxylic acid ethers, phosphoric acid esters and salts, acyl isethionates, alkylaryl sulfonates, alkyl sulfonates, sulfosuccinates, alkyl ether sulfates and alkyl sulfates.

Emollients like Guerbet alcohols and esters thereof, silicone derivatives, beeswax, C12-15 alcohols, benzoate, mineral oil, capric triglycerides, cetearyl alcohol, ceteareth-20, castor oil, isohexadecane, isopropyl myristate, isopropyl palmitate, cetearyl octanoate and petrolatum.

In preparing the final products, polar solvents, such as water, glycols and alcohols, may be used to prepare the cosmetic or coating compositions. The optional alcohol employed in the composition is an aliphatic straight or branched chain monohydric alcohol having 2 to 4 carbon atoms. The concentration of the alcohol in the composition may be less than about 40% by weight, and surprisingly can be as low as 0%, preferably 0-30% by weight.

The compositions of this invention, that is, a cationic material complexed with a compatibilizing agent and an anionic polymer, may be used in a variety of end-use products. Personal care products include: Hair conditioners, styling gels, hair sprays, mousses, shampoos, after shaves, foam baths, hair dyes, shine enhancers, creams, lotions, facial cleansers, skin gels, liquid soaps, body washes, shaving creams, sun care lotions, sun oils, hand cleansers, hand sanitizers, blushes, eye makeups, foundations, concealers, lipsticks and lip balms.

Other products include: Hydroalcoholic gels, wax emulsions, asphalt emulsions, cutting fluids, metal oxide suspensions, mastic adhesives, drawing compound, metalworking fluids, paint strippers, polishes for cars, boats, furniture, leather, marble, silver or chrome, wallpaper removers, liquid fire retarders, solder masks, lapping compounds, automatic dishwashing gels, hand dishwashing liquids or pastes, laundry liquid, laundry prespotter, glass cleaners, hard surface cleaners, abrasive cleaners, sanitizing gels, fragrance gels, and air deodorizers.

### EXPERIMENTAL

### Example 1

PART A: A 6.67 g sample of Avalure® AC-120 polymer, a 30% active acid functional acrylate copolymer from Noveon, was dispersed in 73.3 g water and neutralized.

PART B: A 0.3 g cetrimonium chloride sample was dispersed in 19.7 g water and neutralized.

PART C: A 0.3 g cetrimonium chloride sample was complexed with 1.75 g dimethicone copolyol sulfate, dispersed in 17.95 g water and neutralized.

White precipitate formed upon the addition of B to A. No precipitate formed upon the addition of C to A.

### Example 2

In this example, Fixate™ G-100 polymer from Noveon which is aminomethylpropanol (AMP) acrylates/allyl methacrylate copolymer was tested as the base anionic.

PART A: A 19 g sample of G-100, 1 g glycerin and 0.3 g DMDM Hydantoin were dispersed in 79.2 g water at pH 6.

PART B: A 0.15 g cetrimonium chloride sample was dispersed in 19.85 g water and neutralized.

PART C: A 0.15 g cetrimonium chloride sample was complexed with 1.5 g dimethicone copolyol sulfate, dispersed in 18.35 g water and neutralized.

White precipitate formed upon the addition of B to A. No precipitate formed upon the addition of C to A.

### Example 3

In this example Fixomer™ A-30 polymer (polymethacrylic acid/acrylamidomethyl propane sulfonic acid), a fixative from ONDEA Nalco, is tested was the base anionic.

PART A: A 3.15 g sample of A-30 was dispersed in 76.85 g water at pH 7.

PART B: A 0.3 g olealkonium chloride sample was dispersed in 19.7 g water and neutralized.

PART C: A 0.3 g olealkonium chloride sample was complexed with 1.75 g dimethicone copolyol sulfate, dispersed in 17.95 g water and neutralized.

White precipitate formed upon the addition of B to A. No precipitate formed upon the addition of C to A.

What is claimed is:

1. A method of compatibilizing an anionic polymer with incompatible cationic materials which method comprises complexing said cationic materials with a compatible anionic complexing agent prior to combining said anionic polymer with the complexed cationic material wherein said anionic complexing agent contains a bulky molecule having an anionic group.

2. A method of claim 1, wherein said bulky molecule in a complexing agent has a molecular weight of at least 1,000.

3. A method of claim 2, wherein said bulky molecule is a polymer.

4. A method of claim 3, wherein said anionic polymer is prepared from a monomer containing an acid moiety selected from the group consisting of carboxyl, sulfate, sulfonate, phosphate and phosphonate.

5. A method of claim 3, wherein said polymeric complexing agent is selected from the group consisting of an acrylic copolymer, polyalkylene glycol, polyvinyl alcohol, polyvinyl acetate, polysaccharide, polyurethane and polysilicones.

6. A method of claim 5, wherein said polymeric complexing agent contains an anionic group selected from carboxylate, sulfonate, sulfate, phosphate and phosphonate groups.

7. A method of claim 6, wherein said polymeric complexing agent is a polysilicone.

8. A method of claim 7, wherein said polysilicone is selected from the structure consisting of: 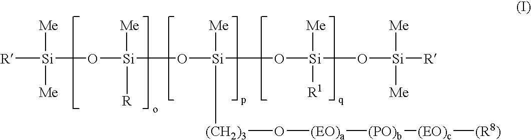
wherein:
Me is methyl;
R and R′ are independently selected from methyl, —OH, —R⁷, and —R⁹—A or —(CH₂)₃—O—(EO)ₐ—(PO)_{b}—(EO)_{c}—G with the proviso that both R and R′ are not methyl, —OH or R⁷;
R¹ is selected from lower alkyl CH₃(CH₂)ₙ— or phenyl where n is an integer from 0 to 22;
a, b, and c are integers independently ranging from 0 to 100;
EO is —(CH₂CH₂O)—; 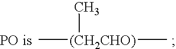
o is an integer ranging from 1 to 200;
q is an integer ranging from 0 to 1000;
p is an integer ranging from 0 to 200;
R⁷ is aryl, alkyl, aralkyl, alkaryl, or alkenyl group of 1-40 carbons;
R⁸ is hydrogen or R⁷ or C(O)—X wherein X is aryl, alkyl, aralkyl, alkaryl, alkenyl group of 1-40 carbons, or a mixture thereof;
R⁹ is divalent group selected from alkylene of 1-40 carbons which may be interrupted with arylene group of 6 to 18 carbons or an alkylene group containing unsaturation of 2 to 8 carbons;
A and G are independently are selected from 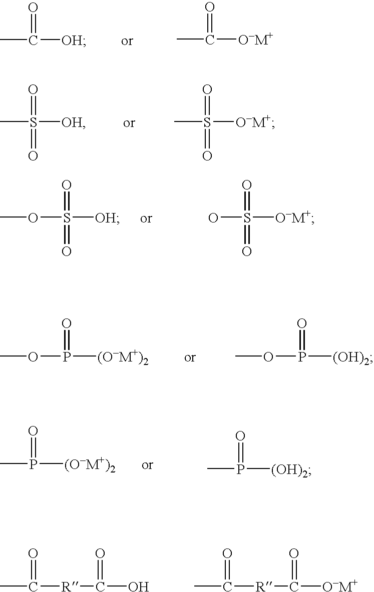
where
R″ is a divalent group selected from alkylene of 1-40 carbons which may be interrupted with an arylene group of 6 to 18 carbons or an alkylene group of 2 to 8 carbons, and is preferably selected from the
R″ is selected from —CH₂—CH₂—; —CH═CH—; 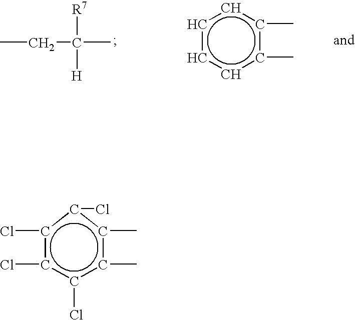
where M is Na, K, Li, NH₄; or an amine containing alkyl, aryl, akenyl, hydroxyalkyl, arylalkyl or alkaryl groups; 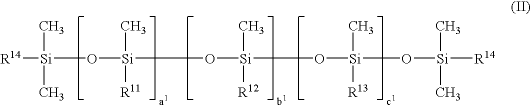
wherein
R¹¹ is selected from lower alkyl having one to eight carbon atoms or phenyl,
R¹² is —(CH₂)₃—O—(EO)ₓ—(PO)_{y}—(EO)_{z}—SO₃⁻M⁺
M is a cation and is selected from Na, K, Li, or NH₄;
x, y and z are integers independently ranging from 0 to 100;
R¹³ is —(CH₂)₃—O—(EO)ₓ—(PO)_{y}—(EO)_{z}—H
R¹⁴ is methyl or hydroxyl;
a¹ and c¹ are independently integers ranging from 0 to 50;
b¹ is an integer ranging from 1 to 50; 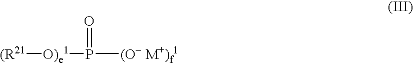
wherein
R²¹ is 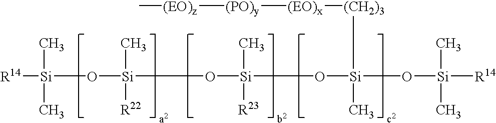
a² is an integer from 0 to 200;
b² is an integer from 0 to 200;
c² is an integer from 1 to 200;
R¹⁴ is as defined above;
R²² is selected from —(CH₂)ₙCH₃ and phenyl;
n is an integer from 0 to 10;
R²³ is —(CH₂)₃—O—(EO)ₓ₁—(PO)_{y1}—(EO)_{z1}—H;
x¹, y¹ ands z¹ are integers and are independently selected from 0 to 20;
e¹ and f¹ are 1 or 2 with the proviso that e+f=3;
M is selected from H, Na, K, Li, or NH₄; and 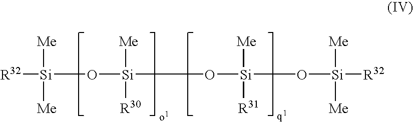
wherein;
Me is methyl;
R³⁰ and R³² independently are —CH₃ or —(CH₂)₃—O—(EO)ₐ3—(PO)_{b}3—(EO)_{c}3—C(O)—R³³—C(O)—OH;
with the proviso that both R³⁰ and R³² are not —CH₃;
R³³ is selected from —CH₂—CH₂—; —CH═CH—; —CH₂—C(R³⁷)—H; 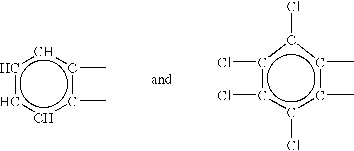
R³⁷ is alkyl having from 1 to 22 carbon atoms;
R³¹ is selected from lower alkyl (having 1-4 carbons), CH₃(CH)ₙ¹— and phenyl;
n¹ is an integer from 0 to 8;
a³, b³ and c³ are integers independently ranging from 0 to 20;
EO is an ethylene oxide residue —(CH₂CH₂—O)—;
PO is a propylene oxide residue —(CH₂CH(CH₃)—O—);
o¹ is an integer ranging from 1 to 200;
q¹ is an integer ranging from 0 to 500.

9. A method of claim 8, wherein said anionic polymer is prepared from a monomer selected from the group consisting of vinyl carboxylic acid, vinyl carboxylic anhydride, vinyl sulfonic acid, vinyl sulfuric acid and vinyl phosphonic acid.

10. A method of claim 6, wherein the anionic polymer is prepared from ethylenically unsaturated monomers at least 10% by weight of which is a monomer containing carboxylic group.

11. A method of claim 10, wherein said polymer contains at least 25% by weight of repeating units derived from a monomer containing carboxylic group.

12. A method of claim 11, wherein the anionic polymer, a rheology modifier is obtained from the polymerization of one or more monomers represented by the formula 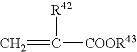
wherein
R⁴³ is hydrogen or an alkyl group having from 8 to 30 carbon atoms and
R⁴² is a substituent selected from the class consisting of hydrogen, halogen, hydroxyl, lactone, lactam and the cyanogens (—CN) groups, monovalent alkyl radicals, monovalent aryl radicals, monovalent aralkyl radicals, monovalent alkaryl radicals and monovalent cycloaliphatic radicals.

13. A method of claim 12, wherein R⁴³ is hydrogen or an alkyl group from 10 to 22 carbon atoms and R⁴² is hydrogen or methyl.

14. A composition of matter comprising an anionic polymer and a cationic material complexed with a compatible anionic complexing agent that contains a bulky molecule having an anionic group.

15. A composition of claim 14 wherein said bulky molecule is a polymer.

16. A composition of claim 15, wherein said anionic polymer is prepared from a monomer containing an acid moiety selected from the group consisting of carboxyl, sulfate, sulfonate, phosphate and phosphonate.

17. A composition of claim 15, wherein said polymeric complexing agent is selected from the group consisting of an acrylic copolymer, polyalkylene glycol, polyvinyl alcohol, polyvinyl acetate, polysaccharide, polyurethane and a polysilicone.

18. A composition of claim 17, wherein said polymeric complexing agent contains an anionic group selected from carboxylate, sulfonate, sulfate, phosphate and phosphonate groups.

19. A composition of claim 18, wherein said polymeric complexing agent is a polysilicone.

20. A composition of claim 19, wherein said polysilicone is selected from the structures consisting of 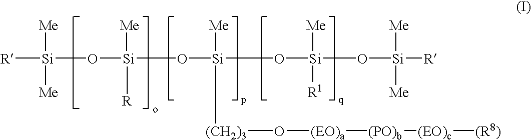
wherein:
Me is methyl;
R and R′ are independently selected from methyl, —OH, —R⁷, and —R⁹—A or —(CH₂)₃—O—(EO)ₐ—(PO)_{b}—(EO)_{c}—G with the proviso that both R and R′ are not methyl, —OH or R⁷;
R¹ is selected from lower alkyl CH₃(CH₂)ₙ— or phenyl where n is an integer from 0 to 22;
a, b, and c are integers independently ranging from 0 to 100;
EO is —(CH₂CH₂O)—; 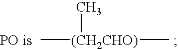
o is an integer ranging from 1 to 200;
q is an integer ranging from 0 to 1000;
p is an integer ranging from 0 to 200;
R⁷ is aryl, alkyl, aralkyl, alkaryl, or alkenyl group of 1-40 carbons;
R⁸ is hydrogen or R⁷ or C(O)—X wherein X is aryl, alkyl, aralkyl, alkaryl, alkenyl group of 1-40 carbons, or a mixture thereof;
R⁹ is a divalent group selected from alkylene of 1-40 carbons which may be interrupted with arylene group of 6 to 18 carbons or an alkylene group containing unsaturation of 2 to 8 carbons;
A and G are independently are selected from 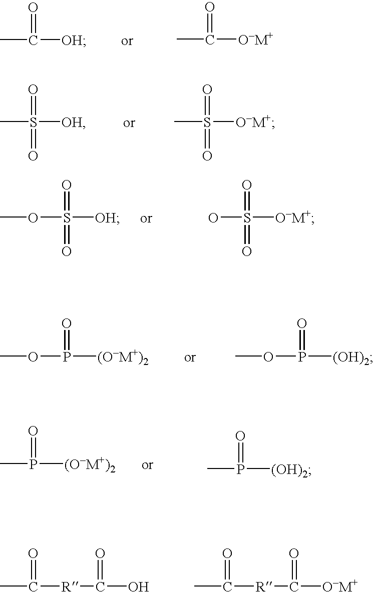
where
R″ is a divalent group selected from alkylene of 1-40 carbons which may be interrupted with an arylene group of 6 to 18 carbons or an alkylene group of 2 to 8 carbons, and is preferably selected from the
R″ is selected from —CH₂—CH₂—; —CH═CH—; 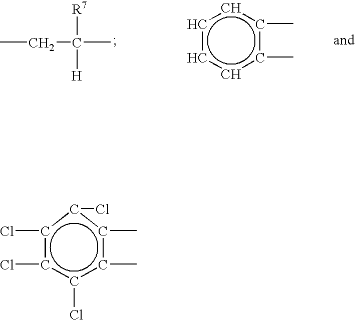
where M is Na, K, Li, NH₄; or an amine containing alkyl, aryl, akenyl, hydroxyalkyl, arylalkyl or alkaryl groups. 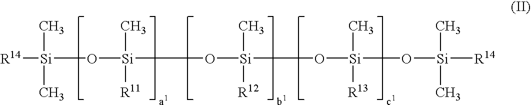
wherein
R¹¹ is selected from lower alkyl having one to eight carbon atoms or phenyl,
R¹² is —(CH₂)₃—O—(EO)ₓ—(PO)_{y}—(EO)_{z}—SO₃^{31 M}⁺
M is a cation and is selected from Na, K, Li, or NH₄;
x, y and z are integers independently ranging from 0 to 100;
R¹³ is —(CH₂)₃—O—(EO)ₓ—(PO)_{y}—(EO)_{z}—H
R¹⁴ is methyl or hydroxyl;
a¹ and c¹ are independently integers ranging from 0 to 50;
b¹ is an integer ranging from 1 to 50; 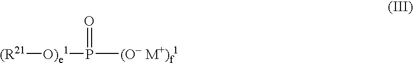
wherein
R²¹ is 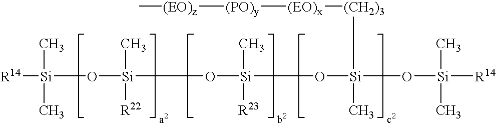
a² is an integer from 0 to 200;
b² is an integer from 0 to 200;
c² is an integer from 1 to 200;
R¹⁴ is as defined above;
R²² is selected from —(CH₂)ₙCH₃ and phenyl;
n is an integer from 0 to 10;
R²³ is —(CH₂)₃—O—(EO)ₓ₁—(PO)_{y1}—(EO)_{z1}—H;
x¹, y¹ ands z¹ are integers and are independently selected from 0 to 20;
e¹ and f¹ are 1 or 2 with the proviso that e+f=3;
M is selected from H, Na, K, Li, or NH₄; and 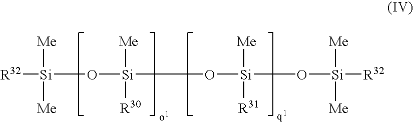
wherein;
Me is methyl;
R³⁰ and R³² independently are CH₃ or —(CH₂)₃—O—(EO)ₐ3—(PO)_{b}3—(EO)_{c}3—C(O)—R³³—C(O)—OH;
with the proviso that both R³⁰ and R³² are not —CH₃;
R³³ is selected from —CH₂—CH₂—; —CH═CH—; —CH₂—C(R³⁷)—H; 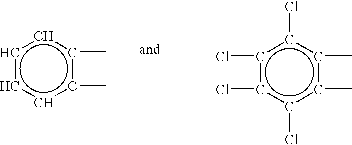
R³⁷ is alkyl having from 1 to 22 carbon atoms;
R³¹ is selected from lower alkyl (having 1-4 carbons), CH₃(CH)ₙ¹— and phenyl;
n¹ is an integer from 0 to 8;
a³, b³ and c³ are integers independently ranging from 0 to 20;
EO is an ethylene oxide residue —(CH₂CH₂—O)—;
PO is a propylene oxide residue —(CH₂CH(CH₃)O);
o¹ is an integer ranging from 1 to 200;
q¹ is an integer ranging from 0 to 500.

21. A composition of claim 20, wherein said anionic polymer is prepared from a monomer selected from the group consisting of vinyl carboxylic acid, vinyl carboxylic anhydride, vinyl sulfonic acid, vinyl sulfuric acid and vinyl phosphonic acid.

22. A coating comprising a composition of claim 14.
